(19) United States
(12) Patent Application Publication
(10) Pub. No.: US 2008/0115791 A1
(43) Pub. Date: May 22, 2008

(54) Mandibular Advancement Mouthpiece, An Intraoccusal Removable Improved Device For Eliminating Or Reducing Snoring

(76) Inventor: Andres Heine, Santiago (CL)

Correspondence Address: BANNER & WITCOFF, LTD., 1100 13th STREET, N.W., SUITE 1200, WASHINGTON, DC, 20005-4051, US

(21) Appl. No.: 11/940,585

(22) Filed: Nov. 15, 2007

(30) Foreign Application Priority Data
Nov. 21, 2006 (CL) 3223-2006

(51) Int. Cl. A61F 5/56

(52) U.S. Cl. 128/848

(57) ABSTRACT

When there is an obstruction to the passage of air through the back of the mouth and nose, the soft palate and the bell collide, producing a vibration called snoring. There are various ways of reducing or eliminating snoring, nasal masks introducing air pressure to the throat (CPAP), surgery of the throat (OPPBA) and devices that are introduced into the mouth to cause a mandibular advancement what increases the oropharyngeal area, improving ventilation, among other solutions. The present invention disclosure an intraocclusal removable device in the form of a “U” that is placed covering all of the upper jaw teeth, wherein two steps, one in each extreme of the lower part of the element, which impede the mandible be closed completely on its normal occlusion, forcing it to produce a forward displacement of the lower jaw.

### DESCRIPTION OF THE PRIOR ART

When there is an obstruction to the passage of air through the back of the mouth and nose, the soft palate and the bell collide, producing a vibration called snoring. There are various ways of reducing or eliminating snoring, nasal masks introducing air pressure to the throat (CPAP), surgery of the throat (OPPBA) and devices that are introduced into the mouth to cause a mandibular advancement what increases the oropharyngeal area, improving ventilation, among other solutions.

The CPAP is uncomfortable to use since the patient should sleep connected to a machine and with a mask. The OPPBA involves an operation and the associated risks and does not always produces satisfactory results. The intraocclusal devices exist, although effective, have a number of disadvantages in terms of discomfort in their use, either by their large size, because they complicate the occlusion, or because they have nuisances elements to the oral sensitivity.

Within these intraocclusal objects there are solutions of two elements, one that is placed in the teeth of the upper jaw and another in the teeth of the mandible and for some system hooks or union, they advance the jaw to create the desired effect of progress. These devices covering both jaw makes them to be too large and is very uncomfortable to use them all night. They also have the effect of immobilize relatively the jaw, impeding the lateral movement with the attendant discomfort. Examples of these are patents registered in the U.S. Pat. Nos. 4,169,473, 5,267,862, 5,365,945, 5,427,117, 5,462,066, 5,499,633, 5,562,108, 5,566,683, 5,601,093, 5,611,1355, 5,642,737, 5,755,219, 5,794,627, 5,823,193, 5,868,138, 5,884,628, 6,053,168, 6,109,265, 6,129,084, 6,170,485, 6,325,064, 6,412,489, 6,450,167, 6,516,805 and 6,604,527 and patents in Europe No. AU 2004100980, AU 2005016547, WO2004054484, CA2389440, EP1203570, US2002000230, AU78280113, FR2816203, WO 0076431, U.S. Pat. No. 6,170,485, U.S. Pat. No. 6,092,523, DE1984468, CA2236503, WO9841175, U.S. Pat. No. 6,450,167, and U.S. Pat. No. 5,941,247.

Other alternatives of these devices do not cover all the teeth, causing difficulties in occlusion and some have elements that are supported in sensitive parts of the oral components, as lips, tongue, or have outgoing elements that cause inconvenience to their use. Sample some patents are the European No. WO2005023158, US 2004094165, and WO03057097, and the U.S. Pat. Nos. 6,295,988 and 6,983,752.

Intraocclusal objects composed of a single element that is positioned on the upper teeth are less bulky and therefore more comfortable to wear. But the solutions so far have not been entirely satisfactory, because they have nuisances elements or rely on the lower incisors, causing pressure on these pieces which manifests itself in pain or unwanted displacements. Examples are patents US 20060196512, U.S. Pat. Nos. 5,092,346 and 6,820,623, and WO200500142 and US2005011525.

The solution to be patent is a intraocclusal removable device which is U-shaped, where one of its faces presents all along a sufficiently wide channel to cover teeth, being the other flat face with two steps one in each of the extremes end of the face, device with the appropriate size to be placed inside the mouth, so that the face where the channel is faces up and this channel covers the teeth of the upper jaw. The steps in the face remain downward, preventing the mouth is closed at its usual occlusion, as resting on last lower molars, and only allow occlusion if the lower jawbone advances so that the steps are positioned on the back of those lower molars, which achieves the desired effect of the mandible progress to increase oropharyngeal space improving ventilation and reducing or to eliminate snoring.

### DESCRIPTION OF THE DRAWINGS

FIG. 1.—represents a an upper perspective of the device.

FIG. 2. represents a lateral perspective of the device.

FIG. 3.—represents a lateral perspective of the device inside the mouth with the jaw without advance.

FIG. 4.—represents a lateral perspective of the device inside the mouth with the advanced jaw.

FIG. 5—Represents perspective of the device with removable steps.

### DESCRIPTION OF THE INVENTION

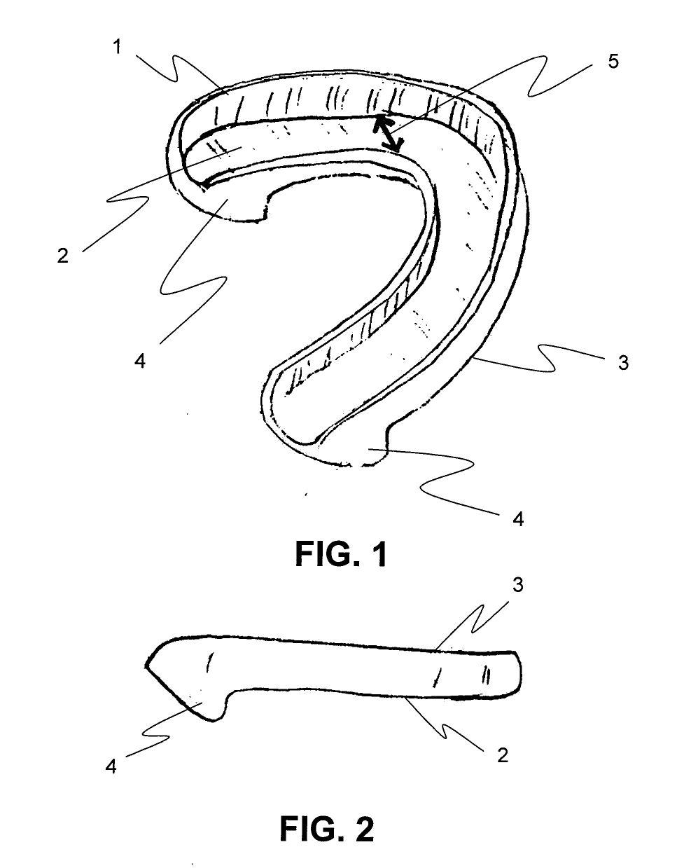

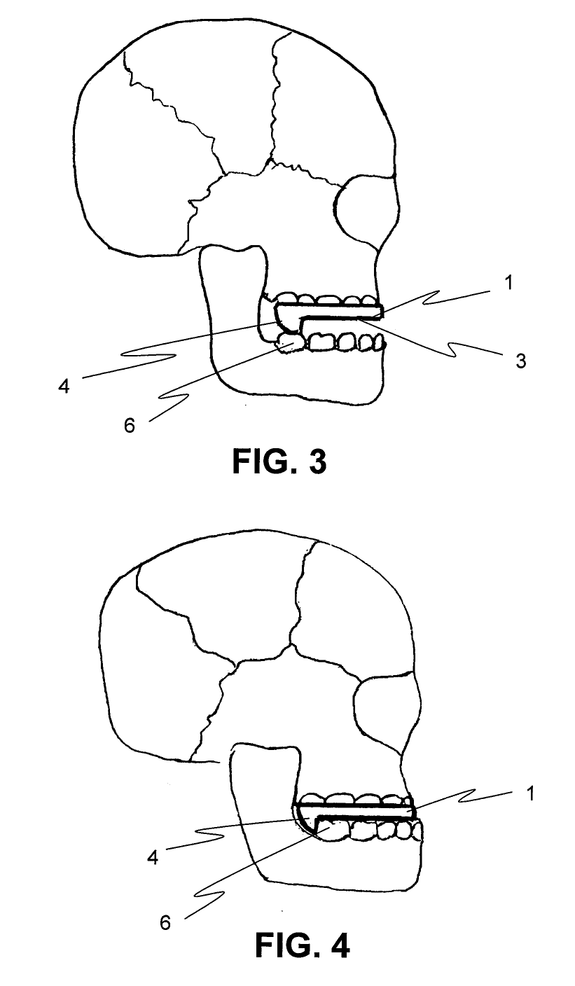

As can be seen in FIG. 1, the invention is a device that is U-shaped (1), where one of their faces (2) goes up and submitted along a channel (5) sufficiently width to fill teeth, and the other side (3), which goes down, flat with two steps (4) that stand one in each of the ends of the face (3). This device is the size appropriate to be placed inside the mouth so that the face (2) where is the channel faces up and this channel covering the teeth of the upper jaw. The way is to use it can be seen in FIG. 3.

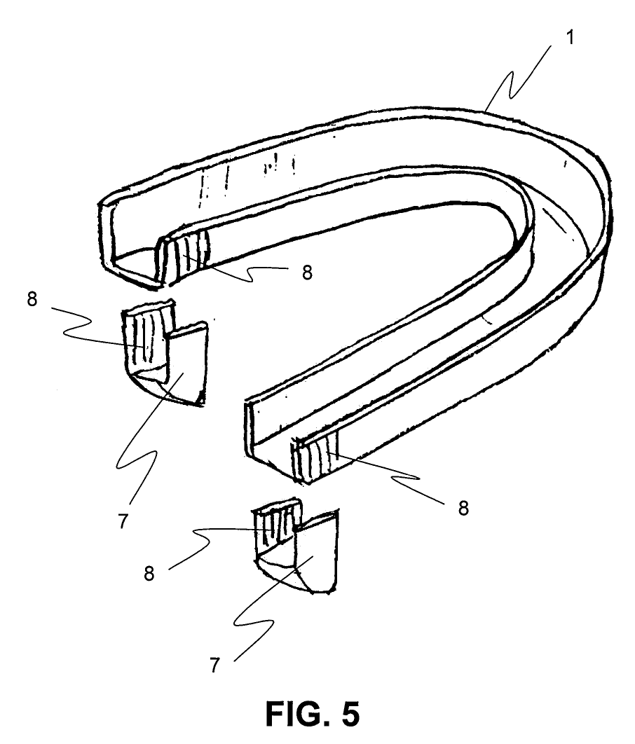

The steps (4) located on the face (3) which remains downward, impede that the mouth is closed at its usual occlusion, as can be seen in FIG. 3, as resting on the last molars lower (7), and only allow occlusion if the jaw forwards so that the treads are positioned on the back of those lower molars, as illustrated in FIG. 4. This achieves the desired effect to progress the mandible and increase the oropharyngeal space, improve ventilation and reduce or eliminate snoring.

As illustrated in FIG. 5, you can also achieve the effect of mandibular advancement if steps that are at the bottom of the plane are of a removable type (6), allowing its placement in different positions, according to the degree of overtaking mandibular there is a desire to achieve. The form of removable hook links may be under pressure and lateral grooves (8) as shown in the picture or other mechanism. This flexibility to provoke a greater or lesser overtaking gives the advantage of going graduating place where the stairs in such a way to adjust the best effect to reduce or eliminate snoring without reaching a mandibular awkward position.

The overtaking can be made in some cases with only one of the two steps, causing the displacement of the mandible. This is particularly advisable in situations where some people will not be able to adequately support on one side of the mandible.

### EXAMPLE

This device can be manufactured at one of its versions preferential acrylic based on a wax mold (or other material) obtained from the upper jaw, adjusting steps so to produce a displacement of the jaw between 4 mm and 8 millimeters, depending of each patient conditions.

1. An intraocclusal removable device in the form of a “U” that is placed covering all of the upper jaw teeth, wherein two steps, one in each extreme of the lower part of the element, which impede the mandible be closed completely on its normal occlusion, forcing it to produce a forward displacement of the lower jaw.

2. An intraocclusal removable device according to claim 1, wherein one step on one extreme of the lower part of the element.

3. An intraocclusal removable device according to claim 1, wherein to take removable steps.

4. An intraocclusal removable device according to claim 1, wherein having at the bottom of the ends of the element, steps in the form of ramp down in a continuous toward the center, producing a pitch forcing keep open the mouth or advance the mandible if desired close, thereby increasing the oropharyngeal area.
